(19) United States
(12) Patent Application Publication
(10) Pub. No.: US 2013/0131607 A1
(43) Pub. Date: May 23, 2013

(54) SET FOR MEDICAL TREATMENT CONTAINING HOLDER FOR CANNULAS

(71) Applicant: Charles Ruben, Stockholm (SE)

(72) Inventor: Charles Ruben, Stockholm (SE)

(21) Appl. No.: 13/811,824

(22) PCT Filed: Aug. 4, 2011
(86) PCT No.: PCT/SE11/00148

§ 371 Date: Jan. 23, 2013

(30) Foreign Application Priority Data
Aug. 10, 2010 (SE) 1000825-8

(51) Int. Cl. A61M 5/32

(52) U.S. Cl. CPC A61M 5/3298
USPC 604/240

(57) ABSTRACT

Set for medical treatment by means of a pen-type holder containing a medical in solution for subkutan injection by means of a disposeable needle or cannula assembly engageable on one end of the pen. The set comprises at least one tubular holder (10) in which new or used cannulas can be inserted and stored the holder having firm fastening means (13) to be detachably adapted to the medical pen or other similar holders (10).

The present invention relates to a set for medical treatment by means of a pen-type holder containing a medical in solution for subkutan injection by means of a disposable needle assembly or cannula engageable to one end of the pen.

Diabetics depending of insulin and supplying insulin via syringe and cannula have to observe to bring the right number of cannulas, apart from the syringe, in such way that neither unused nor used cannulas come in the wrong hands. That could be a risk of infection, find its way to misuse etc, which makes a complement to current inadequate handling necessary.

Further the risk should be eliminated that diabetics forget to bring cannulas when leaving home and insulin is to be injected elsewhere.

The purpose of the invention is thus to guarantee that the proper number of cannulas is brought along when the insulin pen is used away from home, that new as well as used cannulas can be stored easy and safe and that also other medicaments, such as tablets may be stored in the set.

These and other objects are achieved in that the invention has obtained the characteristic features mentioned in the following claims.

The invention will in the following be disclosed in greater detail with reference to embodiments illustrated in the drawings.

FIG. 1 shows a side view of a holder for cannulas.

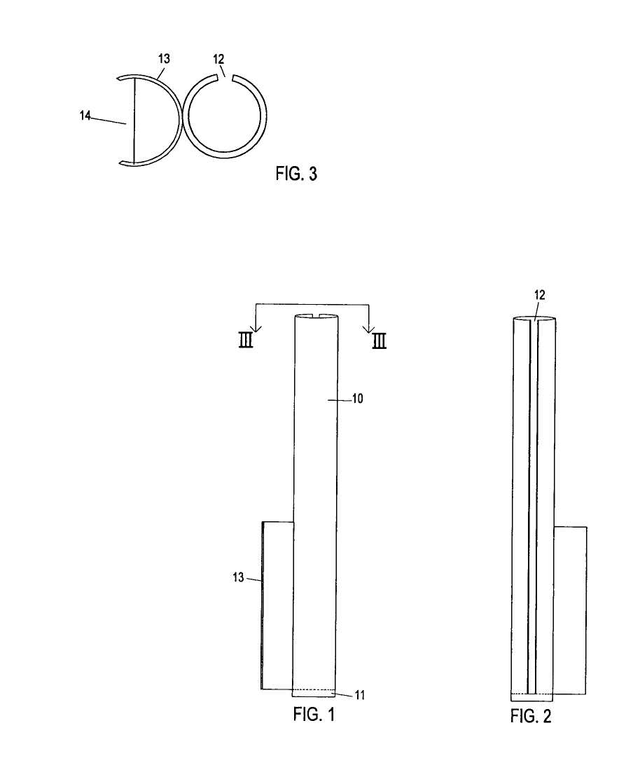

FIG. 2 shows a view of the opposite side of the holder in FIG. 1.

FIG. 3 shows a view of the holder in FIG. 1 seen from above along the line in FIG. 1.

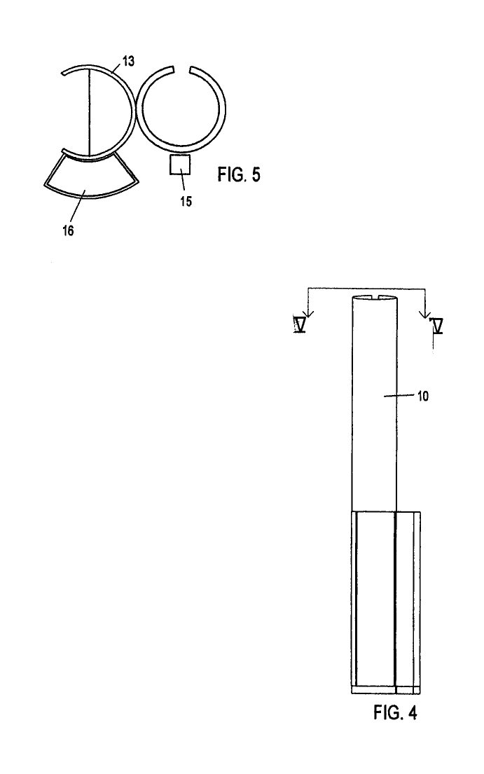

FIG. 4 shows a side view of a altered embodiment of the holder.

FIG. 5 shows a view from above of the holder in FIG. 4 along the line V-V in FIG. 4.

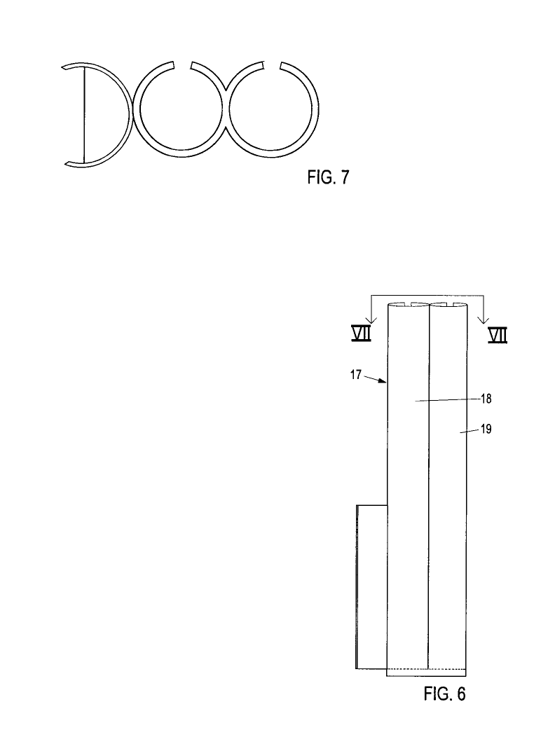

FIG. 6 shows another altered embodiment of the holder.

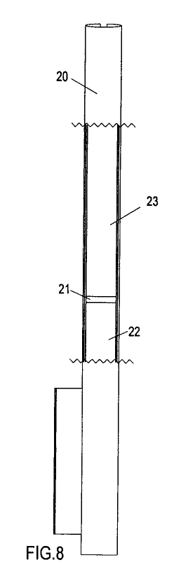

FIG. 7 shows a view from above of the holder in FIG. 6 along the line VII-VII in FIG. 6.

FIG. 8 shows a further embodiment of the cannula holder.

The cannula holder shown in FIG. 1 comprises a tubular body 10 with an inner diameter which allows cannulas to be fed downwards and stored in the tube which at the lower end is closed by a bottom 11. As shown in FIG. 2 the body 10 has a score 12 so that the number of cannulas stored in the tube can be read. A clip 13 is at the lower part of one side of the holder 10 adapted to retain a medical pen (not shown) or a further cannula holder, the insertion of which in the clip 13 is hindered by a stop such as a traverse pin or thread 14. The holder 10 has also upwards a lid (not shown) of suitable type.

In the embodiment shown in FIGS. 4 and 5 the holder 10 is provided with a pocket clip 15 and at the clip 13 a holder 16 is firmly fixed for storing medicals in tablet form, which holder is provided with a suitable lid (not shown).

In FIGS. 6 and 7 is shown a twin holder 17 manufactured with two united tubular bodies 18, 19 and provided with a clip for holding a medical pen as has been described before. Here can also if needed be provided a tablet holder as shown before.

FIG. 8 shows a side view partly sectioned of a holder 20, which by means of a partition wall 21 is divided into two separate rooms 22 and 23 which are reached from both ends of the holder 20 which are open for insertion of cannulas in the holder 20 and can be closed with suitable lids.

It is obvious from the above that the holder 10 in FIG. 1 simply by the help of the clip 13 can be attached to a syringe, for instance an insulin pen, whereby the holder 10 is filled with unused cannulas. By fastening a further holder 10 to the first one, used cannulas can be collected in this further holder.

Alternatively the twin holder 17 shown in FIG. 6 and FIG. 7 can be used together with a medical pen. Due to the simple fastening of the holders by means of clips, any wanted number and combination of holders can of course be brought about within the scope of the invention.

It is thus obvious that the invention is not limited to the embodiments shown and described but changes can be made in order to adapt the components to individually desired ends. Thus pocket clip and tablet holder may be arranged on other sides of the cannula holder and clip than those shown.

1. Set for medical treatment by means of a pen-type holder containing a medical in solution for subkutan injection by means of a disposeable needle assembly or cannula engageable on one end of the pen, characterized in that the set comprises at least one tubular holder (10) in which cannulas can be inserted and stored, the holder having firm fastening means (13) to be detachable adapted to the medical pen or other similar holders (10), the fastening means being formed as a clip (13) arranged to springy encircle the medical pen or other holders.

2. Set according to claim 1, characterized in that the holder is manufactured of two united tubular bodies (18, 19) to form a twin holder (17).

3. Set according to claim 1-2, characterized in that at the holder (10, 17) a tablet holder (16) for medicin in tablet form is arranged.

4. Set according to any of claims 1-3, characterized in that the holder (20) by means of a partition wall (21) is divided into two separate rooms (22, 23).

5. Set according to any of claims 1-4, characterized in that the holder (10) is provided with a pocket clip (15).

6. Set according any of claims 1-5, characterized in that the cannula holder is provided with a score (12) so that the number of cannulas stored in the holder (10) can be read.
